SEQUENCE LISTING (10 sequences)

SEQ ID NO: 1 - DNA, 60 bp, Artificial Sequence
  Synthetic Sequence
ctgtaagataatgttgctttcttatcccagtgatcacctgccaaatgaataagacaacaa

SEQ ID NO: 2 - DNA, 32 bp, Artificial Sequence
  Synthetic Sequence
agtgatcacctgccaaatgaataagacaacaa

SEQ ID NO: 3 - DNA, 32 bp, Artificial Sequence
  Synthetic Sequence
ggtgatcacctgccaaatgaataagacaacaa

SEQ ID NO: 4 - DNA, 32 bp, Artificial Sequence
  Synthetic Sequence
tgtgatcacctgccaaatgaataagacaacaa

SEQ ID NO: 5 - DNA, 32 bp, Artificial Sequence
  Synthetic Sequence
cgtgatcacctgccaaatgaataagacaacaa

SEQ ID NO: 6 - DNA, 60 bp, Artificial Sequence
  Synthetic Sequence
acgtaggaaaatgtgaaatgttcctgttcttacataaaagaactctcagaaaatacccgt

SEQ ID NO: 7 - DNA, 31 bp, Artificial Sequence
  Synthetic Sequence
atacataaaagaactctcagaaaatacccgt

SEQ ID NO: 8 - DNA, 31 bp, Artificial Sequence
  Synthetic Sequence
gtacataaaagaactctcagaaaatacccgt

SEQ ID NO: 9 - DNA, 31 bp, Artificial Sequence
  Synthetic Sequence
ttacataaaagaactctcagaaaatacccgt

SEQ ID NO: 10 - DNA, 31 bp, Artificial Sequence
  Synthetic Sequence
ctacataaaagaactctcagaaaatacccgt

(19) United States
(12) Patent Application Publication
(10) Pub. No.: US 2007/0238106 A1
(43) Pub. Date: Oct. 11, 2007

(54) Systems and methods of determining alleles and/or copy numbers

(75) Inventors: Michael T. Barrett, Mountain View, CA (US); Nicholas M. Sampas, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Loveland, CO (US)

(21) Appl. No.: 11/400,532

(22) Filed: Apr. 7, 2006

(51) Int. Cl. C12Q 1/68; C12M 3/00

(52) U.S. Cl. 435/6; 435/287.2; 977/924

(57) ABSTRACT

Various aspects of the invention include a solid support having a first region with a first nucleic acid, and second, third, fourth and fifth regions having, respectively, second, third, fourth, and fifth nucleic acids each having a length that is shorter than the length of the first nucleic acid. Certain aspects of the invention further include embodiments where the second, third, fourth, and fifth nucleic acids are substantially identical, and no two of the second, third, fourth, and fifth nucleic acids ends with identical terminal nucleotides. Some aspects of the invention further include methods of using the solid support, and kits that include the same.

### BACKGROUND

Many genomic and genetic studies are directed to the identification of differences in gene dosage or expression among cell populations for the study and detection of disease. For example many cancers and premalignant lesions typically contain multiple regions of gains or losses of genomic DNA sequences resulting in activation of oncogenes or inactivation of tumor suppressor genes. Identification of the genetic events leading to neoplastic transformation and subsequent progression can facilitate efforts to define the biological basis for disease, improve prognostication of therapeutic response, and permit earlier tumor detection. In a number of other diseases, including autism, and mental retardation, the genomic copy numbers of various genomic intervals are important to the disease states and their treatment. In addition, developmental disorders, such as autism and mental retardation syndromes, are associated with losses or gains of genomic segments up to and including whole chromosomes. Thus, methods of pre and postnatal detection of such abnormalities can be helpful in early, accurate diagnosis and management of these conditions.

Comparative genomic hybridization (CGH) is one approach that has been employed to detect the presence and identify the location of amplified or deleted sequences. In one implementation of CGH, genomic DNA is isolated from normal reference cells, as well as from test cells (e.g., tumor cells). The two nucleic acids are differentially labeled and then hybridized in situ to a reference cell, e.g., to metaphase chromosomes. Chromosomal regions in the test cells which are at increased or decreased copy number can be identified by detecting regions where the ratio of signal from the two DNAs is altered. For example, those regions that have been decreased in copy number in the test cells will show relatively lower signal from the test DNA than the reference compared to other regions of the genome. Regions that have been increased in copy number in the test cells will show relatively higher signal from the test DNA.

In a recent variation of the above traditional CGH approach, the immobilized chromosome element has been replaced with a collection of solid support bound target nucleic acids, e.g., an array of BAC (bacterial artificial chromosome) clones or cDNAs. Such approaches offer benefits over immobilized chromosome approaches, including a higher resolution, as defined by the ability of the assay to localize chromosomal alterations to specific areas of the genome. However, these methods still have significant limitations in their ability to detect chromosomal alterations at single gene resolution (in the case of BAC clone arrays) or in non-coding regions of the genome in the case of cDNA clone arrays. In addition, array features containing longer lengths of nucleic acid sequence are more susceptible to binding cross-hybridizing sequences, where a given immobilized target nucleic acid hybridizes to more than one distinct probe sequence. This property limits somewhat the ability of these technologies to detect low level amplifications and deletions sensitively and accurately.

In another recent variation, a CGH platform has been developed that can detect genomic aberrations, including single copy losses, homozygous deletions, as well as amplicons of variable sizes throughout the human genome using non-reduced complexity samples of genomic DNA as targets, as discussed in M. T. Barrett, et al., “Comparative Genomic Hybridization using Oligonucleotide Microarrays and Total Genomic DNA,” Proc. Natl. Acad. Sci. USA, 101(51):17765-17770 (2004), incorporated herein by reference. Other variations include those discussed in U.S. patent application Ser. No. 10/448,298, filed May 28, 2003, entitled “Comparative Genomic Hybridization Assays using Immobilized Oligonucleotide Targets with Initially Small Sample Sizes and Compositions for Practicing the Same,” by M. T. Barrett, et al., published as U.S. Patent Application Publication No. 2004/0241658 on Dec. 2, 2004; or International Patent Application No. PCT/US2003/041047, filed Dec. 22, 2003, entitled “Comparative Genomic Hybridization Assays using Immobilized Oligonucleotide Features and Compositions for Practicing the Same,” by L. K. Bruhn, et al., published as WO 2004/058945 A2 on Jul. 15, 2004; each of which is incorporated herein by reference.

In addition to genomic lesions that result in copy number changes, cancer genomes can contain allelic alterations (e.g., nondysjunction, gene conversion) that occur in the absence of copy number changes of genomic material. The latter category of lesions is frequently associated with loss of heterozygosity (LOH) events and can have profound phenotypic effects on a genome. However, they cannot be easily detected using CGH. Accordingly, there is a need for improved methods of genetic analytical techniques, including improvements to CGH assays.

### SUMMARY OF THE INVENTION

The present invention generally relates to techniques involving comparative genomic hybridization, including systems and methods of determining alleles and/or copy numbers in a target nucleic acid. The subject matter of the present invention involves, in some cases, interrelated products, alternative solutions to a particular problem, and/or a plurality of different uses of one or more systems and/or articles.

In one aspect, a solid support is provided. In one set of embodiments, the solid support comprises, in a first region, a first nucleic acid; and in second, third, fourth, and fifth regions, respectively, second, third, fourth, and fifth nucleic acids. In some cases, each has a length that is shorter than the length of the first nucleic acid, and in certain embodiments, each is immobilized relative to the solid support at a first end of each respective nucleic acid. In some instances, each of the second, third, fourth, and fifth nucleic acids is substantially identical. In one embodiment, no two of the second, third, fourth, and fifth nucleic acids ends with identical terminal nucleotides.

In another set of embodiments, the solid support comprises, in first, second, third, fourth, and fifth regions, respectively, first, second, third, fourth, and fifth nucleic acids. In some cases, each of the second, third, fourth, and fifth nucleic acids is substantially identical. In one embodiment, each of the second, third, fourth, and fifth nucleic acids has between 20 nucleotides and 50 nucleotides, inclusive. In certain instances, the first nucleic acid comprises a portion substantially identical to the second, third, fourth, and fifth nucleic acids.

In another aspect, a kit is provided, comprising a first nucleic acid, and second, third, fourth, and fifth nucleic acids. In some cases, each has a length that is shorter than the length of the first nucleic acid, and in certain instances, each of the second, third, fourth, and fifth nucleic acid being substantially identical. In one set of embodiments, no two of the second, third, fourth, and fifth nucleic acids ends with identical terminal nucleotides.

In yet another aspect, a method is provided, comprising acts of providing a first nucleic acid; providing at least three types of substantially identical nucleic acid probes, each having a length that is shorter than the length of the first nucleic acid no two of the at least three types of substantially identical nucleic acid probes ending with identical terminal nucleotides; hybridizing a target nucleic acid to at least some of the nucleic acid probes, the target nucleic acid having a length greater than the length of the nucleic acid probes; substantially complementarily extending one of the probe nucleic acids along the target nucleic acid, without substantially extending the length of the other probe nucleic acids by subjecting the probe nucleic acids to primer extension reaction conditions; evaluating binding of the target nucleic acid and the first nucleic acid; and evaluating binding of the target acid with the nucleic acid probes.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C schematically illustrate an assay to determine the allele and the copy number of a target nucleic acid, according to one embodiment of the invention;

FIG. 2 shows an example of a substrate carrying an array, in accordance with one embodiment of the invention;

FIG. 3 shows an enlarged view of a portion of FIG. 2; and

FIG. 4 shows an enlarged view of another portion of the substrate of FIG. 2.

### BRIEF DESCRIPTION OF THE SEQUENCES

SEQ ID NO: 1 is CTGTAAGATAATGTTGCTTTCTTATCCCAGTGAT
CACCTGCCAAATGAATAAGACAACAA, an example of a CGH
probe;
SEQ ID NO: 2 is AGTGATCACCTGCCAAATGAATAAGACAACAA,
an example of an A-allelic probe;
SEQ ID NO: 3 is GGTGATCACCTGCCAAATGAATAAGACAACAA,
an example of a G-allelic probe;
SEQ ID NO: 4 is TGTGATCACCTGCCAAATGAATAAGACAACAA,
an example of a T-allelic probe;
SEQ ID NO: 5 is CGTGATCACCTGCCAAATGAATAAGACAACAA,
an example of a C-allelic probe;
SEQ ID NO: 6 is ACGTAGGAAAATGTGAAATGTTCCTGTTCTTACA
TAAAAGAACTCTCAGAAAATACCCGT, an example of a CGH
probe;
SEQ ID NO: 7 is ATACATAAAAGAACTCTCAGAAAATACCCGT,
an example of an A-allelic probe;
SEQ ID NO: 8 is GTACATAAAAGAACTCTCAGAAAATACCCGT,
an example of a G-allelic probe;
SEQ ID NO: 9 is TTACATAAAAGAACTCTCAGAAAATACCCGT,
an example of a T-allelic probe;
and
SEQ ID NO: 10 is CTACATAAAAGAACTCTCAGAAAATACCCGT,
an example of a C-allelic probe.

### DETAILED DESCRIPTION

DNA molecules are present within all living cells. DNA encodes genetic instructions that define the composition, construction and functions of each cell. By “processing” these instructions, the cell can produce certain proteins or molecules, and/or perform various activities. Genomic DNA is comprised of sets of chromosomes, and each chromosome is a long, polymeric molecule in which the genetic information is encoded by combinations of the four “natural bases,” or nucleotides (adenine, guanine, cytosine, and thymine) or molecular units, in each position along the DNA. This is roughly analogous to “beads on a string,” where a string may have a large number of beads on it, encoding various types of information, although each bead along the string can only be of one of four different colors.

However, there are differences between the DNA of any two individuals, or even among the copies of sequences in the same individual. In many cases these differences are present within an individual “gene” (essentially, a unit of information encoded within the DNA) and are as subtle as a single base variation. A single base difference is often referred to as a “SNP” (which stands for “single nucleotide polymorphism”), typically pronounced “snip.” Knowledge of these differences is important for certain applications, such as the study of natural variation, cancer research, or research into hereditary diseases.

Moreover, in some instances, there may also be errors in the DNA. These errors may arise, for example, in various types of cancer. One example of a common error is the accidental duplication of one or more genes within the DNA. For example, in normal human cells, a gene typically appears as two copies within the genome. Scientists refer to this as diploid state. Each copy is obtained from either the maternal or paternal genome and is referred to as an allele of the gene of interest. In cancerous genes, however, the gene may appear zero, one, two, three, four, or more times within the DNA (i.e., a copy number of 0, 1, 2, 3, or 4, respectively).

Scientists would like to be able to determine both the copy number and the SNP content (i.e. allele) of a given gene or sequence within a given DNA molecule. However, there have previously been no adequate techniques for detecting both of these characteristics. This invention discloses several novel techniques.

In an example of one of these techniques, a substrate is prepared having two distinct types of probe molecules attached to it, where the probe molecules recognize certain base sequences of the DNA, and can bind to the DNA to form a “complex” of the DNA and the probe. One type of probe molecule is relatively long, and, when bound to the DNA, can be used to indicate the copy number of the DNA. The other type of probe molecules are a series of 4 probe molecules, shorter than the first type of probe molecule, that are all substantially identical, with the exception of the terminal base. Here, each of the 4 types of probes ends with a different base. While the DNA can bind to each of the 4 probes, only one of the DNA molecules can subsequently be “lengthened” using certain types of chemical reactions, based on the terminal base of the DNA probe, as discussed in detail below. Determination of which DNA molecule was lengthened can then be used to determine the SNP content of the DNA sample. Thus, by using both types of probes, e.g., on the same surface, both the copy number and the allele of a gene or region of interest within a given DNA molecule.

More specifically, the present invention generally relates to techniques involving comparative genomic hybridization, including systems and methods of determining alleles and copy numbers in a set of target nucleic acids. In one aspect, the invention is directed to an assay able to determine the copy number and the allelic variation of each target nucleic acid, for example, that arises from a genome. In one embodiment of the invention, a first nucleic acid probe is provided that can be used to determine copy number of the nucleic acid, and a set of second nucleic acid probes is provided that can be used to determine the allele. Each of these may be attached to a surface, such as the surface of an array. In some cases, the first nucleic acid probe is similar to a CGH (comparative genomic hybridization) probe, and may be used to determine copy number of the target nucleic acid. The set of second nucleic acid probes may be at least substantially identical in some cases. For instance, there may be four types of nucleic acids, each of which are identical except that each is terminated with a different terminal nucleotide. Exposure of the set of second nucleic acid probes to target nucleic acids may result in specific hybridization of the complementary sequences present in the target nucleic acids to some or all of the probes. The resulting probe-target sequence duplexes are then subjected to conditions that promote the extension of probes along the length of the complementary target sequence. Those probes perfectly complementary to a target sequence can be extended in such a fashion, while other probes, such as those with a mismatch at their terminal nucleotides, will not be efficiently extended. Accordingly, by identifying those probes that have been extended, the allele or alleles present within the mixture of target nucleic acids can be determined.

The assays of the present invention allow for the determination of acquired (e.g., somatic) variations in the alleles and the copy numbers of target nucleic acids in a given sample (e.g., a cancer genome) relative to a normal reference, and for germ-line (e.g. constitutive variations (e.g., SNPs and copy number polymorphisms) present in an individual or population.

The method can be used with reduced complexity genomes, or non-reduced complexity genomes in some cases, to determine the copy number and the allele. The genome may arise from any suitable genomic source. A “non-reduced complexity genome” is a genome in which the complexity has not been reduced in some fashion, e.g., the genome is in a native state. The “non-reduced complexity” collection of nucleic acids, is compared to the initial genomic source, genomic template and genome of the organism from which the initial genomic source is obtained. A non-reduced complexity collection of nucleic acids is one that is produced in a manner designed to retain the complexity of the sample nucleic acids, e.g., is not produced using collections of primers that are designed to prime only a certain percentage or fraction of the initial genomic source. For example, a reduced complexity collection of nucleic acid targets is one that has been produced by a protocol that preferentially amplifies certain portions, fractions, or regions of the genomic source material during the preparation and the collection of target sequences.

In certain embodiments, a non-reduced complexity collection of nucleic acid targets is one in which a substantial fraction, if not all, of the sequences or subsequences of the initial genomic source (and organism genome from which the initial source is obtained) are represented by the sequences of the target sequence population. By “substantially all,” it is meant typically at least about 50%, such as at least about 60%, at least about 70%, at least about 75%, at least about 80%, at least about 85%, at least about 90% or more, including at least about 95%, at least about 97% etc, of the total genomic sequences are present in the produced target population, where the above percentage values are number of bases in the produced target population as compared to the total number of bases in the genomic source. Because substantially all, if not all, of the non-repetitive sequences found in the genomic source are present in the produced population of target nucleic acids, the resultant population of target nucleic acids is not one that is reduced in complexity with respect to the initial genomic template, i.e., it is not a reduced complexity population of target nucleic acids.

A non-reduced complexity collection of nucleic acid targets can be readily identified using a number of different protocols. One comprehensive protocol for determining whether a given collection of nucleic acid targets is a non-reduced complexity collection of nucleic acid targets is to screen the collection using a genome wide array of target nucleic acids for the genomic source of interest. Thus, one can tell whether a given collection of nucleic acid targets has non-reduced complexity with respect to its genomic source by assaying the collection with a broad genomic array that includes probes for both the targeted regions and non-targeted genomic loci. The genome-wide array of the genomic source is an array of target nucleic acids in which the entire genomic source is screened at a sufficiently high resolution, where the resolution is typically at least about 1 Mb, e.g., at least about 500 kb, such as at least about 250 kb, including at least about 100 kb, e.g., 50 kb or higher (such as 25 kb, 15 kb, 10 kb or higher), where resolution in this context means lengths of the genomic source between regions present on the array in the form of immobilized targets. In such a genomic wide assay of sample, a non-reduced complexity sample is one in which substantially all of the features on the array hybridize to specific complementary sequences present in the target sample, where by substantially all is meant at least about 50%, such as at least about 60%, 70%, 75%, 80%, 85%, 90%, or 95% (by number) or more.

A “reduced complexity genome” is a genome in which the complexity has been reduced using various PCR and other genome complexity reduction methods known to those of ordinary skill in the art. The reduced complexity genome can, under ideal circumstances, provide a reliable representation of a specific region or regions of the whole genome, and can be analyzed as though it were a genome of considerably lower complexity.

The complexity of the nucleic acid probes may be, for instance, at least about 20-fold less, at least about 25-fold less, at least about 50-fold less, at least about 75-fold less, at least about 90-fold less, or at least about 95-fold less, than the complexity of the initial genomic source, in terms of total numbers of sequences found in the produced population of probes as compared to the initial source, up to and including a single gene locus being represented in the collection. The reduced complexity can be achieved in a number of different manners, such as by using gene specific primers in the generation of labeled nucleic acid probes, by reducing the complexity of the genomic source used to prepare the nucleic acid probes, etc. As with non-reduced complexity protocols, in these reduced complexity protocols, the nucleic acid probes prepared in many (but not all) embodiments are labeled nucleic acid probes. Any convenient labeling protocol, such as the above described representative protocols, may be employed, where the protocols are adapted to provide for the desired reduced complexity, e.g., by using gene specific instead of random primers.

Thus, in one embodiment, the entire genome of an organism may be used, and the target nucleic acid may be prepared from the entire genome, while in another embodiment, the genome of the organism may be reduced in complexity, prior to preparing the target nucleic acid. In some embodiments, the collection or population of nucleic acid probes that is prepared is one that is labeled with a detection entity, as discussed herein.

The method can distinguish various lesions present in cancerous genomes, for example, allelic alterations (e.g., nondysjunction, gene conversion, allele-specific amplification, etc.) that cannot be resolved by either CGH or allelotyping alone. In some cases, these changes may occur in the absence of copy number changes of genomic material. Some changes, like loss of heterozygosity, may also be associated either with copy number changes or without, but these different manifestations may be indicative of different phenotypes. In addition to detecting loss of heterozygosity, the ability to discriminate alleles in regions of copy number alterations may provide increased information regarding the phenotypic consequences of allelic variants in tumorigenesis and human diseases. For example, identifying allelic variations of selected genes or haplotypes present in amplicons may help to identify alleles that confer elevated risk in normal populations. One example is a single nucleotide polymorphism (SNP). SNPs are widespread throughout the human (there are an estimated 10 million) and other mammalian genomes and show sufficient variability among individuals in a population that they have become important in several fields including genetic mapping, linkage analysis, and human identity testing. Furthermore, they can be used to detect and map regions of somatic allelic loss and imbalances in cancer genomes.

The combination of CGH assays and allele assays yields several unexpected advantages. As an example, the use of a common array surface for CGH and allelotyping probes reduces error in simultaneously determining alleles and copy numbers, Additionally, the combination assay eliminates several steps involved with preparing a target nucleic acid for analysis. Moreover, the two assays do not substantially interfere with each other, as discussed below.

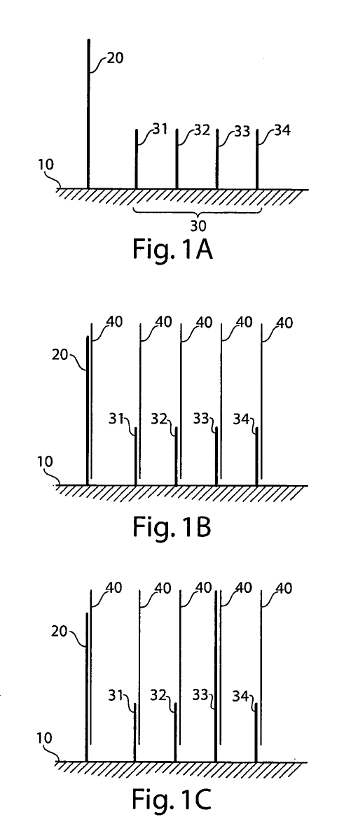

A non-limiting example of an assay according to one embodiment of the invention is illustrated in FIG. 1. In FIG. 1A, immobilized relative to substrate 10 is a nucleic acid probe 20. Nucleic acid probe 20 is bound to the substrate at one end, e.g., at the 3′ end of the nucleic acid probe. It should be noted that although only one molecule of each type of nucleic acid or nucleic acid probe is depicted in the figures, this is for purposes of clarity only. In actuality, there may be multiple, identical copies of each type of nucleic acid or nucleic acid probe present.

In some embodiments of the invention, the nucleic acid probe is a comparative genomic hybridization (CGH) probe, which can be used to determine the copy number of the target nucleic acid. Typically, the CGH probe is chosen to be perfectly complementary, or at least substantially complementary, to the target nucleic acid, such that hybridization of the target nucleic acid to the CGH probe can be used to determine copy number of the target nucleic acid. Alternatively multiple CGH probes, each complementary to a known or putative allele for any given target sequence of interest is present on the array.

Also immobilized relative to substrate 10 is a set 30 of nucleic acid probes useful for determining alleles and allelic variations. Set 30 may include more than one type of nucleic acid probe. For example, in FIG. 1A, set 30 is depicted as having four types of allele nucleic acid probes 31, 32, 33, and 34, each terminated with a different nucleotide or nucleotide analog. The nucleic acid probes may each be immobilized at the 3′ ends in some instances. Typically, the set of nucleic acid probes are chosen to be substantially complementary to the target nucleic acid, and hybridization efficiency of the target nucleic acid to the nucleic acid probes can be used to determine the allele of the target nucleic acid, as discussed in detail below under a set of conditions that may or may not be the same as the CGH probe.

In certain cases, the nucleic acid probes used for determining alleles are identical or substantially identical to each other, except that the unbound end of each of the nucleic acid probes is different. For example, each of allele nucleic acid probes 31, 32, 33, and 34 may be substantially identical, except that nucleic acid probe 31 ends in an adenine residue (“A”), nucleic acid probe 32 ends in a guanine residue (“G”), nucleic acid probe 33 ends in a cytosine residue (“C”), and nucleic acid probe 34 ends in a thymine residue (“T”). Additionally, nucleic acid probes 31, 32, 33, and 34 may each be substantially identical to a portion of first nucleic acid 20, for example, the portion of nucleic acid 20 closest to substrate 10. Thus, for example, each of nucleic acid probes 20, 31, 32, 33, and 34 may be substantially identical for at least those portions of the nucleic acid probes in closer proximity to the surface of substrate 10.

The nucleic acid probes may be immobilized relative to the substrate in any suitable order or relative orientation. Substrate 10 may be, for example, an array, in which nucleic acid probes 20 and each of nucleic acid probes 31, 32, 33, and 34 are present in different, predetermined locations on the array.

As schematically depicted in FIG. 1B, substrate 10 is then exposed to a target nucleic acid 40, e.g., a genomic target. As each of nucleic acid probes 20, 31, 32, 33, and 34 are substantially complimentary to at least a portion of target nucleic acid 40, target nucleic acid 40 is able to hybridize to each of the nucleic acid probes under suitable conditions, for example, under relatively low stringency conditions (e.g., 37° C. for a short period of time, for instance, less than 30 minutes). Thus, in FIG. 1B, target nucleic acid 40 is at least substantially complementary to nucleic acid probe 20, while a smaller portion of target nucleic acid 40 is substantially complementary to nucleic acid probes 31, 32, 33, and 34, thus resulting in the hybridization of target nucleic acid 40 to each of nucleic acid probes 20, 31, 32, 33, and 34.

Next, substrate 10 is subjected to conditions which allow the growth and extension of the nucleic acid probes. For example, substrate 10 may be exposed to a polymerase under suitable conditions, typically under conditions in which deoxynucleotide substrates such as dATP, dCTP, dGTP, and dTTP (deoxyadenosine triphosphate, deoxycytidine triphosphate, deoxyguanosine triphosphate, and deoxythymidine triphosphate, respectively) are present to facilitate the polymerase reaction at temperatures and buffer conditions that optimize the polymerization.

Under these conditions, if two nucleic acids are hybridized, but are overlapping such that the 3′-end of one nucleic acid overhangs the 5′-end of the other nucleic acid, the polymerase is able to “extend” the non-overhanging nucleic acid at its 5′-end until either a mismatch is encountered or its end is in alignment with the 3′-end of the second nucleic acid. Such extension conditions are well-known in the art. However, such extension typically occurs only if there are no mismatches on the growing end(s) of the two nucleic acids.

Thus, although target nucleic acid 40 is substantially complementary to each of allele nucleic acid probes 31, 32, 33, and 34 in set 30, only one of the allele nucleic acid probes is complementary at its terminal to each allele in the target mixture end with respect to nucleic acid 40. Thus, only the nucleic acid probes of set 30 that are perfectly complementary to target nucleic acid 40 can be extended in such a fashion. For homozygous samples, this will typically be a single probe sequence, and for heterozygous samples this will typically be two distinct probe sequences.

As a non-limiting example, in FIG. 1C, allele nucleic acid probe 33 is perfectly complementary to nucleic acid 40 at the 5′-terminal end of the probe, and thus, allele nucleic acid probe 33 can be extended along the length of target nucleic acid 40 when substrate 10 is exposed to suitable conditions which allow the growth and extension of the nucleic acid probes. In contrast, the remaining allele nucleic acid probes 31, 32, and 34 are not complementary at their terminal ends to target nucleic acid 40, and cannot be extended in such a fashion. Thus, allele nucleic acid probes 32, 32, and 34, do not become extended with respect to nucleic acid probe 40.

In some cases, target nucleic acid 40 may be separately hybridized to nucleic acid probe 20, e.g., if the hybridization of target nucleic acid 40 to the allele nucleic acid probes is performed under conditions that are not favorable to hybridizing target nucleic acid 40 to nucleic acid probe 20. For example, high stringency conditions may be used to facilitate such hybridization (e.g., greater than 65° C. and/or longer periods of time, such as at least an hour).

Next, substrate 10 is assayed (e.g., quantitatively) to determine the level of hybridization of nucleic acid 40 with each of nucleic acid probes 20, 31, 32, 33, and 34, e.g., using template dependent primer extension reaction conditions. With respect to first nucleic acid probe 20 (e.g., a CGH probe) and target nucleic acid 40, their hybridization can be analyzed, for instance, using fluorescent labels or other suitable detection entities, as further described herein. For example, in some cases, the target nucleic acid may be labeled with one or more detection entities, and in some embodiments, the detection entities may be distinguishable from detection entities that may be used with respect to the allele nucleic acid probes. For instance, the target nucleic acid probe may be labeled with a single fluorochrome (e.g., one-color hybridization), separately with two different fluorochromes (e.g., two-color hybridization, e.g., for comparison to a reference), etc. By associating the fluorescence of the fluorochrome(s) with nucleic acid probe 20, characteristics such as the degree of similarity or the copy number can be determined. Such techniques are discussed in more detail below.

With respect to allele nucleic acid probes 31, 32, 33, and 34 and target nucleic acid 40, their hybridization can be analyzed, for instance, using fluorescent labels or other suitable detection entities, as further described herein. In some cases, detection entities can be attached to target nucleic acid 40 (which detection entity may be the same or different as that described above with respect to nucleic acid probe 20) prior to hybridization of the allele nucleic acid probes 31, 32, 33, and 34 and the target nucleic acid. The detection entities may be used, for example to determine which (if any) of nucleic acid probes 31, 32, 33, and 34 have been extended, and thus, the identity of the terminal nucleotide or nucleotides at the end of the original allele nucleic acid probes. Such information can then be used to determine the identity of the complementary nucleotide within target nucleic acid 40, thus giving the allele each distinct of target nucleic acid 40 (e.g., a SNP within the target nucleic acid), as discussed in more detail herein.

As used herein, the term “determining” generally refers to the analysis of a species, for example, quantitatively or qualitatively, and/or the detection of the presence or absence of the species. “Determining” may also refer to the analysis of an interaction between two or more species, for example, quantitatively or qualitatively, and/or by detecting the presence or absence of the interaction.

The target nucleic acid to be probed (e.g., DNA 20 in FIG. 1) may be any nucleic acid, for example, DNA or RNA, and the nucleic acid may arise from any suitable source, for example, genomic DNA, cDNA, synthetic DNA, or the like. Target nucleic acids can be derived from virtually any source. Typically, the target nucleic acids will be nucleic acid molecules having sequences derived from representative locations along a chromosome of interest, a chromosomal region of interest, an entire genome of interest, a cDNA library, or the like. The target nucleic acid may have any suitable length. For example, the nucleic acid may have a length of at least 40 nucleotides, at least about 50 nucleotides, at least about 75 nucleotides, at least about 100 nucleotides, at least about 200 nucleotides, at least about 300 nucleotides, etc. As other examples, the nucleic acid may have a length ranging from about 10 nucleotides to about 200 nucleotides, including from about 10 nucleotides or about 20 nucleotides to about 100 nucleotides, from about 50 nucleotides to about 90 nucleotides, about 50 nucleotides to about 80 nucleotides, or about 50 nucleotides to about 70 nucleotides. In some cases, for example, with genomic DNA, the nucleic acid may optionally first be cleaved, for instance, using chemicals or restriction endonucleases known to those of ordinary skill in the art. Typically, the target nucleic acid to be probed is single stranded. Where double-stranded nucleic acids are used, e.g., in the case of double-stranded DNA, the double-stranded nucleic acid may be melted or denatured prior to, or simultaneously with, hybridization of the probe to the device. In the case of double stranded DNA, only those probes bound to support at their 3′-ends are viable for extension.

In certain aspects of the invention, a set of nucleic acid probes are prepared for a desired target, e.g., locus and/or gene or exon of interest, e.g., to determine a SNP or other allele of interest on a nucleic acid, such as a genomic target. For example, a set of nucleic acid probes may include a full-length CGH probe and a set of four allele-specific probes, optionally attached to a surface, such as the surface of an array, as discussed herein.

The first nucleic acid probe may be a comparative genomic hybridization (CGH) probe. The CGH probe can be used to detect certain types of the genomic aberrations in a target nucleic acid, for example, single copy losses, homozygous deletions, as well as amplicons of variable copy numbers or sizes within a target nucleic acid, e.g., a genomic target. Examples of techniques for preparing and using CGH probes have been disclosed in U.S. patent application Ser. No. 10/448,298, filed May 28, 2003, entitled “Comparative Genomic Hybridization Assays using Immobilized Oligonucleotide Targets with Initially Small Sample Sizes and Compositions for Practicing the Same,” by M. T. Barrett, et al., published as U.S. Patent Application Publication No. 2004/0241658 on Dec. 2, 2004; International Patent Application No. PCT/US2003/041047, filed Dec. 22, 2003, entitled “Comparative Genomic Hybridization Assays using Immobilized Oligonucleotide Features and Compositions for Practicing the Same,” by L. K. Bruhn, et al., published as WO 2004/058945 on Jul. 15, 2004; and U.S. patent application Ser. No. 10/744,595, filed Dec. 22, 2003, entitled “Comparative Genome Hybridization Assays using Immobilized Oligonucleotide Features and Compositions for Practicing the Same,” by L. Bruhn et al., published as U.S. Patent Application Publication No. 2004/0191813 on Sep. 30, 2004, each incorporated herein by reference. Additionally, techniques for identifying and preparing suitable CGH probes, given a nucleic acid target, are disclosed in U.S. patent Ser. No. ______, filed ______ under docket number 10040115-01, entitled “Probe Design Methods and Microarrays for Comparative Genomic Hybridization and Location Analysis,” by Sampas, et al., also incorporated herein by reference.

The CGH probe may be of any suitable length. For example, the length of the CGH probe may be between 30 nucleotides and 200 nucleotides, inclusive, between 35 nucleotides and 100 nucleotides, between 40 nucleotides and 80 nucleotides, or between 45 nucleotides and 60 nucleotides. Probes having such nucleotide lengths may be prepared using any suitable method, for example, using de novo DNA synthesis techniques known to those of ordinary skill in the art, such as solid-phase DNA synthesis techniques, or U.S. patent application Ser. No. 11/234,701, filed Sep. 23, 2005, entitled “Methods for In Situ Generation of Nucleic Acid Molecules,” incorporated herein by reference. In some embodiments, the CGH probe is immobilized with respect to a surface of a substrate. For instance, the CGH probe may be immobilized at the 3′ end of the CGH probe, with the 5′ end of the CGH probe being furthest away from the surface of the substrate.

Many methods for immobilizing nucleic acids, such as CGH probes, on a surface are known in the art. The desired component may be covalently bound or noncovalently attached through nonspecific binding, adsorption, physisorption, or chemisorption. If covalent bonding between a compound and the surface is desired, the surface may include appropriate functionalities to provide for the covalent attachment. Functional groups which may be present on the surface and used for linking can include, but are not limited to, carboxylic acids, aldehydes, amino groups, cyano groups, ethylenic groups, hydroxyl groups, mercapto groups and the like. The manners of linking a wide variety of compounds to various surfaces are well known and is amply illustrated in the literature. For example, methods for immobilizing nucleic acids by introduction of various functional groups to the molecules are known (see, e.g., Bischoff et al., Anal. Biochem. 164:336-344 (1987); or Kremsky et al., Nuc. Acids Res. 15:2891-2910 (1987)). Modified nucleotides can be placed on the target using PCR primers containing the modified nucleotide, or by enzymatic end labeling with modified nucleotides, or by non-enzymatic synthetic methods.

Covalent attachment of the target nucleic acids to glass or synthetic fused silica can be accomplished according to a number of known techniques. Such substrates provide a very low fluorescence substrate, and a highly efficient hybridization environment. There are many possible approaches to coupling nucleic acids to glass that employ commercially available reagents. For instance, materials for preparation of silanized glass with a number of functional groups are commercially available or can be prepared using standard techniques. Alternatively, quartz cover slips, which have at least 10-fold lower auto fluorescence than glass, can be silanized. In certain embodiments of interest, silanization of the surface is accomplished using the protocols described in U.S. Pat. No. 6,444,268, the disclosure of which is herein incorporated by reference, where the resultant surfaces have low surface energy that results from the use of a mixture of passive and functionalized silanization moieties to modify the glass surface, i.e., they have low surface energy silanized surfaces. Additional linking protocols of interest include, but are not limited to: polylysine as well as those disclosed in U.S. Pat. No. 6,319,674, the disclosure of which is herein incorporated by reference. The targets can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith et al. Science, 258:1122-1126 (1992)). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

The CGH probe may be used to determine the copy number of a nucleic acid, e.g., a genomic target. As used herein, “copy number” is given its ordinary meaning as used in the art, i.e., the number of times a certain nucleic acid sequence appears within a genome. The copy numbers of regions within a genome are altered by events that amplify or delete sequences or subsequences within the genome. Variations in copy number detectable by the methods of the invention may arise in different ways. For example, copy number may vary as a result of amplification or deletion of a chromosomal region, e.g. as commonly occurs in cancer. Other variations are germ line genomic differences that are inherited through ancestors. Still other de novo variations arise spontaneously during mitosis or meiosis. Techniques for determining the copy number of the target nucleic acid are discussed in more detail below.

The target nucleic acid may be hybridized to the CGH probe under any suitable conditions. Suitable conditions for hybridizing nucleic acid sequences, at least a portion of which are substantially complimentary are known to those of ordinary skill in the art. For example, suitable denaturing agents, or salt and/or buffer solutions in which to perform the hybridization reaction may be readily identified without undue effort. Typically, the hybridization is performed under conditions in which the target nucleic acid to be probed is single-stranded. Where double-stranded nucleic acids are used, e.g., in the case of double-stranded DNA, the double-stranded nucleic acid may be melted or denatured prior to, or simultaneously with, hybridization of the probe and the target nucleic acid.

For example, in one set of embodiments, hybridization of the target nucleic acid and the CGH probe may occur under high stringency conditions. A “high stringency condition,” as used herein, is given its ordinary meaning as used in the art, e.g., conditions in which two nucleic acids having fully complementary portions are able to hybridize, but two nucleic acids having a certain amount of nucleotide mismatch are not able to hybridize. For example, two nucleic acids that are substantially complementary but have mismatches of greater than 5%, 10%, or 15% (by number) may not be able to hybridize under high stringency conditions. Those of ordinary skill in the art will be aware of suitable high stringency conditions useful in causing two nucleic acids having substantially complementary portions to hybridize. For example, the temperature of the solution containing the nucleic acids may be increased to at least about 50° C., at least about 55° C., at least about 60° C., at least about 65° C., at least 70° C., etc. for a suitable period of time to allow hybridization to take place, for example, at least about 1 hour, at least about two hours, at least about three hours, etc. In some cases, high stringency conditions include conditions that are compatible to produce binding pairs of nucleic acids and nucleic acid probes of sufficient complementarity to provide for the desired level of specificity in the assay while being incompatible to the formation of binding pairs between binding members of insufficient complementary to provide for the desired specificity. Stringent conditions are discussed in greater detail below.

In some embodiments, a reference nucleic acid may be used simultaneously with the target nucleic acid, e.g., to determine copy number, allele, or other characteristics of the target nucleic acid. The reference nucleic acid is a sequence believed to be identical or at least substantially similar to the target nucleic acid, but which sequence is already known. In some cases, the reference nucleic acid comprises a detection entity; in other cases, however, the reference nucleic acid does not comprise a detection entity. For example, in one set of embodiments, the target nucleic acid comprises a first detection entity and the reference nucleic acid comprises a second detection entity different from the first detection entity. By exposing the nucleic acids simultaneously to a CGH probe and determining the degree to which the first detection entity and/or the second detection entity is associated with the CGH probe, characteristics of the target nucleic acid, such as its copy number, can be determined. As other examples, the target nucleic acid may be unlabeled and the reference nucleic acid may be labeled with a detection entity, the target nucleic acid may be labeled with a detection entity and the reference nucleic acid may be unlabeled, etc. After exposure of both to the CGH probe, the association of the detection entity and the CGH probe may be determined to determine characteristics of the target nucleic acid, e.g., its copy number.

As used herein, a “detection entity” is an entity that is capable of indicating its existence in a particular sample or at a particular location. One non-limiting example of a detection entity is a fluorescent moiety. Detection entities of the invention can be those that are identifiable by the unaided human eye, those that may be invisible in isolation but may be detectable by the unaided human eye if in sufficient quantity, entities that absorb or emit electromagnetic radiation at a level or within a wavelength range such that they can be readily detected visibly (unaided or with a microscope including a fluorescence microscope or an electron microscope, or the like), spectroscopically, or the like. Non-limiting examples include fluorescent moieties (including phosphorescent moieties), radioactive moieties, electron-dense moieties, dyes, chemiluminescent entities, electrochemiluminescent entities, enzyme-linked signaling moieties, etc. In some cases, the detection entity itself is not directly determined, but instead interacts with a second entity (a “signaling entity”) in order to effect determination; for example, coupling of the signaling entity to the detection entity may result in a determinable signal. The detection entity may be covalently attached to the nucleic acid probe as a separate entity (e.g., a fluorescent molecule), or the detection entity may be integrated within the nucleic acid, for example, covalently or as an intercalation entity, as a detectable sequence of nucleotides within the nucleic acid probe, etc. In some embodiments, the nucleic acid probe is immobilized with respect to the detection entity in a manner that does not reduce the complexity to any significant extent as compared to the initial genomic source. For instance, a number of different nucleic acid labeling protocols are known in the art and may be employed to produce a population of labeled nucleic acid probes. The particular protocol may include the use of labeled primers, labeled nucleotides, modified nucleotides that can be conjugated with different dyes, one or more amplification steps, etc.

In some cases, the target nucleic acid is hybridized to the CGH probes simultaneously with hybridization of the target nucleic acid to the allele probes, as further discussed below. However, in other embodiments, the various hybridization steps do not necessarily occur at the same time. For example, hybridization of the target nucleic acid to the CGH probe may occur before or after hybridization of the target nucleic acid to the allele probes.

The association of the target nucleic acid (and/or a reference nucleic acid, if present in the same assay) and a CGH probe can be determined using any suitable technique. Non-limiting examples of suitable techniques for assaying a CGH probe has been disclosed in U.S. patent application Ser. No. 10/448,298, filed May 28, 2003, entitled “Comparative Genomic Hybridization Assays using Immobilized Oligonucleotide Targets with Initially Small Sample Sizes and Compositions for Practicing the Same,” by M. T. Barrett, et al., published as U.S. Patent Application Publication No. 2004/0241658 on Dec. 2, 2004; or International Patent Application No. PCT/US2003/041047, filed Dec. 22, 2003, entitled “Comparative Genomic Hybridization Assays using Immobilized Oligonucleotide Features and Compositions for Practicing the Same,” by L. K. Bruhn, et al., published as WO 2004/058945 A2 on Jul. 15, 2004; each of which is incorporated herein by reference.

In one set of embodiments, the target nucleic acid can be labeled with one or more detection entities, which may be associated with and/or incorporated within the target nucleic acid and/or the reference nucleic acid. By determining the presence or absence of the detection entity, or the amount and/or concentration of the detection entity with respect to the CGH probe, the degree of association of the target nucleic acid with the CGH probe can be determined. For example, in a competitive assay involving a target nucleic acid comprising a first detection entity and a reference nucleic acid comprising a second detection entity different from the first detection entity, by determining the ratio of the first detection entity with respect to the second detection entity, relative to the CGH probe, the ratio of binding of the target nucleic acid with respect to the reference nucleic acid can be determined. Thus, the ratios of the copy number of genomic sequences of interest present in the first target relative to the second target can be determined. As another example, a target nucleic acid may comprise a first detection entity while a reference nucleic acid does not comprise a detection entity, and the amount and/or concentration of the first detection entity relative to the CGH probe may be determined to determine the ratio of binding of the target nucleic acid with respect to the reference nucleic acid. In yet another example, the target nucleic acid may not comprise a detection entity while a reference nucleic acid comprises a detection entity. In still another example, binding of the target nucleic acid may be determined using a first assay, and separately, binding of a reference nucleic acid may be determined using a second assay. The target nucleic acid and the reference nucleic acid may comprise the same or different detection entities, and the amount of binding of each nucleic acid may be compared in some fashion, e.g., by determining the association of the detection entities with the CGH probes.

By determining the association of the target nucleic acid and the CGH probe, information such as copy number, gene duplication, amplification, etc. of the target nucleic acid may be determined. For instance, variations in the copy number, relative to the reference nucleic acid, may indicate overrepresentation or underrepresentation of a target nucleic acid, e.g., a gene. A cell could thus be determined to be tumorous, or a patient could be diagnosed with a cancer or other disease involving aberrant development or uncontrolled cell proliferation. As an example, if the target nucleic acid and the reference nucleic acid have the same copy number, then the relative amounts of association with the CGH probe would be expected to be about equal. Conversely, higher or lower copy numbers may be indicated by differences in the relative amounts of detection entities detected. For example, higher amounts of the target nucleic acid, relative to the reference nucleic acid, may be indicative of high copy numbers; while lower amounts of detection entity may be may be indicative of low copy numbers. Those of ordinary skill in the art are able to quantify such copy number determinations, using only routine technique, for example, constructing a calibration curve involving multiple reference samples, each of which has a known copy number.

Such a determination of copy number of a target nucleic acid may be performed, in some cases, within the same sample (i.e., a reference nucleic acid and the target nucleic acid are provided together in a sample, each having a different detection entity immobilized relative thereto, and determining the relative amounts of binding between the target and the referenced nucleic acids to the CGH probe. However, in other cases, the target and the reference nucleic acids may be brought to two different CGH probes, i.e., on different substrates, and the relative amounts of each that become immobilized with respect to the surface are compared. In yet other cases, the target and the reference nucleic acids can be brought into the same sample, but are exposed to two different CGH probes that have been immobilized to a surface. Besides CGH probes, one or more types of nucleic acid probes useful for determining an allele (i.e., an allele probe) can also be used, for example, immobilized with respect to a surface of a substrate. For instance, the allele probe may be immobilized at the 3′ end of the allele probe, with the 5′ end of the allele probe being furthest away from the surface of the substrate.

In some embodiments, the copy numbers of particular nucleic acid sequences in two probe collections are compared by hybridizing the probes to one or more target nucleic acids, as described above. The hybridization signal intensity, and/or the ratio of intensities, produced by the probes on each of the target elements can be determined. Since signal intensities on a target element can be influenced by factors other than the copy number of a probe in solution, for certain embodiments, an analysis may be conducted where two labeled populations are present with distinct labels. Thus, comparison of the signal intensities for a specific target element may permit direct comparison of copy numbers of the two samples for a given sequence. Different target elements will reflect the copy numbers for different sequences in the probe populations. The comparison can reveal, for instance, situations where each sample includes a certain number of copies of a sequence of interest, but the numbers of copies in each sample are different. The comparison can also reveal situations where one sample is devoid of any copies of the sequence of interest, and the other sample includes one or more copies of the sequence of interest.

Typically, a plurality of different allele probes are used, which are often identical or substantially identical to each other, with the exception that the terminal nucleotide of each allele probe is different for each type of allele probe, i.e., the nucleotide on the 5′ end of the allele probe. For instance, three or four allele probes may be used, each of which are substantially identical, but differ by a small number of nucleotides, for example, 3, 2, or 1 nucleotide (i.e., the terminal nucleotide). For example, the nucleic acid probes may be substantially identical, except that none of the probe types ends with identical terminal nucleotides, i.e., one allele probe may end with adenine (A), one may end with guanine (G), one may end with cytosine (C), and one may end with thymine (T). Thus, in one embodiment, no two of the nucleic acid probes ends with identical terminal nucleotides.

In some embodiments, the allele probes are basically a shortened version of the CGH probe. The allele probes may be chosen to be long enough to bind a target nucleic acid sufficiently during the extension portion of the assay, yet sufficiently short to inhibit stable duplexes during CGH hybridization. The allele probe and the CGH probe may each have the same 3′ ends (e.g., attached to a substrate), but differ in their lengths. The allele probes are typically shorter than the CGH probe. For example, the allele probes may have a length of between 20 nucleotides and 50 nucleotides, inclusive, between 20 nucleotides and 40 nucleotides, between 20 nucleotides and 35 nucleotides, etc. In one set of embodiments, the allele probes are shorter than the CGH probe. For instance, the allele probe may have a length that is less than about three-fourths, less than about two-thirds, or less than the length of the CGH probe.

In one set of embodiments, the allele to be detected is a single nucleotide polymorphism (SNP). The SNP may also be referred to as a “polymorphic base” in some instances. A SNP is a DNA sequence variation that occurs when a single nucleotide within a genome is altered. In some cases, the SNP occurs within the coding sequence of a gene and is a point mutation, i.e., it alters the amino acid that is translated from the gene sequence. In other cases, however, the SNP within the coding sequence of a gene does not alter the amino acid that is expressed, i.e., due to the redundancy of the Genetic Code. Detection of SNPs is often important in determining variations in population or genealogy, or in determining the presence of allelic variations associated with tumor cells. In some cases, SNPs may also be used as a marker of a disease. SNPs are widespread throughout the human and other mammalian genomes and show sufficient variability among individuals in a population that they have become important in some fields including genetic mapping, linkage analysis, and human identity testing. Further, they can be used to detect and map regions of somatic allelic loss and imbalances in cancer genomes.

In one set of embodiments, the allele probes may be prepared using a computer sorting technique. CGH assays are frequently carried out under conditions in which the target nucleic acids are fragmented by means of a restriction digest involving one or more restriction enzymes. For this reason CGH probes are typically designed such that they do not include restriction cleavage sites, but may they may be terminated at either end by such a restriction cleavage site. For example, the technique may include acts of selecting a target site of a genome, locating a SNP site of the genome in the proximity of the target site, for example within about 100,000 nucleotides of the target site, determining a restriction site between 20 and 35 nucleotides of the SNP site, and preparing a nucleic acid having between 20 and 35 nucleotides and being substantially identical to the genome between the restriction site and the SNP site. Thus, given a target gene or location of interest, one of ordinary skill in the art will be able to identify a SNP corresponding to the gene of interest, and prepare a suitable allelic probe able to detect that SNP.

The target nucleic acid may be hybridized to the allele probes under any suitable conditions, for example, as previously discussed, and those of ordinary skill in the art will be able to identify suitable conditions. In one set of examples, hybridization of the target nucleic acid and the allele probes may occur under “low stringency conditions,” i.e., conditions in which two nucleic acids having portions that are at least substantially complementary are able to hybridize with reasonable specificity. For example, a low stringency condition may be selected such that nucleic acids will hybridize reasonably efficiently where the target nucleic acid are probe nucleic acid are substantially complementary and contiguous over at least 35 nucleotides, at least 30 nucleotides, at least 20 nucleotides, at least 15 nucleotides, etc. As another example, the nucleic acids may hybridize even if there are mismatches of greater than 5%, 10%, 15%, 20%, 25%, 30%, 35%, or 40% (by number) between the two nucleic acids. Those of ordinary skill in the art will be aware of suitable low stringency conditions useful in causing two nucleic acids to hybridize.

As an example, in a low stringency condition, the temperature of the solution containing the nucleic acids may be brought to a temperature of less than about 50° C., less than about 45° C., less than about 40° C., less than about 37° C., etc., but sufficient to allow hybridization to occur. Such temperatures may be maintained for a period of time at least sufficient to allow hybridization to occur. In some cases, hybridization may occur relatively rapidly. For example, times of less than about two hours, less than about one hour, or less than about 30 minutes may be sufficient in some cases to allow hybridization of the target nucleic acid and the allele probes.

In some cases, the target nucleic acid is hybridized to the allele probes simultaneously with hybridization of the target nucleic acid to the CGH probes. However, in other embodiments, the various hybridization steps do not necessarily occur at the same time. For example, hybridization of the target nucleic acid to the allele probes may occur before or after hybridization of the target nucleic acid to the CGH probes.

During or after hybridization of the target nucleic acid to the allele nucleic acid probes, the allele nucleic acid probes may be exposed to conditions which allow the growth and extension of the probes from its 5′-end. For example, the allele nucleic acid probes may be exposed to a suitable polymerase enzyme, in conjunction with deoxynucleotide substrates which can be polymerized through action of the polymerized to the allele nucleic acid probes. Examples of polymerases include, but are not limited to, Taq, Pwo, Pfu, Vent, Deep Vent, Tfl, HotTub, Tth, Klenow fragment, Phi29, etc, which are to known to those of ordinary skill in the art and are readily available. The polymerase may be chosen to be one with high specificity, i.e., the polymerase is able to extend an allele nucleic acid probe, using the target nucleic acid as a template to produce a perfectly complementary nucleic acid, only if the allele nucleic acid probe and the polymerases are initially perfectly complementary within the footprint of the enzyme, for example within the first 8 bases from the 5′-end of the probe nucleic acid. For example, if the allele nucleic acid probe and polymerase are substantially complementary, with the exception of the terminal nucleotide of the nucleic acid probe, then the polymerase may be selected to be unable to extend the allele nucleic acid probe, using the target nucleic acid as a template, after the point of mismatch. Accordingly, if all of the allele nucleic acid probes were chosen to be identical or substantially identical, with the exception of the terminal nucleic acid of each probe, then only one of the allele nucleic acid probes can be extended in such a complementary fashion, i.e., the one which has the correct nucleic type that is perfectly complementary to the target nucleic acid. Thus, as is shown in FIG. 1C, only one of the allele nucleic acid probes can be extended under these conditions.

Those of ordinary skill in the art will be able to identify suitable conditions which allow the growth and extension of a nucleic acid along a template nucleic acid (e.g., the target nucleic acid), e.g. through action of a polymerase, in conjunction with suitable deoxynucleotide substrates. In some cases, extension of an allele nucleic acid probe occurs simultaneously with hybridization of the target nucleic acid to the allele nucleic acid probes; thus, the conditions described above can also be used to facilitate extension of the allele nucleic acid probe through action of the polymerase. In some cases, growth and extension of the nucleic acid may occur relatively rapidly. For example, times of less than about two hours, less than about one hour, or less than about 30 minutes may be sufficient in some cases to allow growth and extension of the allele nucleic acid probe using the target nucleic acid as a template. However, in other embodiments, the allele nucleic acid probes are not exposed to the polymerase simultaneously with the exposure of the allele nucleic acid probes to the polymerase.

In some embodiments, a reference nucleic acid may be used simultaneously with the target nucleic acid to determine the allele of the target nucleic acid. Binding of the target nucleic acid to the allele nucleic acid probes may be compared with binding of the reference nucleic acid to the allele nucleic acid probes to determine the amount of binding of the target nucleic acid with the allele nucleic acid probes, and/or which of the allele nucleic acid probes has been extended, as further discussed below.

As discussed above, the reference nucleic acid is a sequence believed to be identical or at least substantially similar to the target nucleic acid, but which sequence is already known. In some cases, the reference nucleic acid comprises a detection entity; in other cases, however, the reference nucleic acid does not comprise a detection entity. For example, in one set of embodiments, the target nucleic acid comprises a first detection entity and the reference nucleic acid comprises a second detection entity different from the first detection entity.

This reference nucleic acid may be the same or different than the reference nucleic acid (if present) used in conjunction with the CGH probe, as previously described. Thus, in one embodiment, a first reference nucleic acid is used to determine copy number and a second reference nucleic acid (which may be the same or different than the first reference nucleic acid) is used to determine the allele of the target nucleic acid. Either or both of the reference nucleic acids may be used in the same assay as the target nucleic acid, or used in different assays and the results compared. In another embodiment, no reference nucleic acid is used to determine copy number, and a reference nucleic acid is used to determine allele. In yet another embodiment, a reference nucleic acid is used to determine copy number, but no reference nucleic acid is used to determine allele. In still another embodiment, no reference nucleic acids are used to determine copy number or allele.

The association of the target nucleic acid (and/or a reference nucleic acid, if present in the same assay) and the allele nucleic acid probes can be determined using any suitable technique. Detection entities may also be used, in some cases, to determine the allele of the target nucleic acid. The detection entity used to determine alleles of the target nucleic acid may be the same or different than the detection entity or entities used to determine immobilization with respect to the CGH probe. The detection entity may be bound to the target nucleic acid using any suitable technique, for example, end labeling or other enzymatic or chemical treatments that do not modify the sequence of the target nucleic acid may be used. Other methods of binding the detection entity to a nucleic acid have been described above.

By determining the presence or absence of the detection entity, or the amount and/or concentration of the detection entity with respect to the allele nucleic acid probes, the association of the target nucleic acid and one or more of the allele nucleic acid probes can be determined, e.g., which of the allele nucleic acid probes has been extended. In some cases, an assay such as a single color assay may be used to determine association of a target nucleic acid with one or more allele nucleic acid probes.

For example, the target nucleic acid and the allele nucleic acid probes may initially be free of a detection entity, and after immobilization of the target nucleic acid and the allele nucleic acid probes, a detection entity able to bind to an extended portion of an allele nucleic acid probes may be used to determine which of the allele nucleic acid probes has been extended. As described above, determination of the extended allele nucleic acid probe can be used to determine the correct allele of the target nucleic acid.

As another example, target nucleic acid and reference nucleic acid extension reactions can be performed using separate assays. A detection entity may be present before the extension reaction, or added after the extension reaction. The amount of detection entity bound to each of the allele nucleic acid probes may then be determined for the target nucleic acid and the reference nucleic acid, and compared to determine which of the allele nucleic acid probes has been extended.

As yet another example, a target nucleic acid may include a first detection entity, and a reference nucleic acid includes a second detection entity different from the first detection entity. Both the target nucleic acid and the reference nucleic acid may be allowed to bind the allele nucleic acid probes, and the relative amounts of immobilization of the first detection entity and the second detection entity determined to determine which of the allele nucleic acid probes has been extended.

In certain aspects, one or more types of nucleic acid probes, such as those described above, may be attached to a surface of a solid support or substrate. The surface may be any suitable surface in which a nucleic acid probe may be attached, for example, the surface of a substrate, the surface of a particle, etc. If more than one nucleic acid probe is used (e.g., a CGH probe and one or more allele nucleic acid probes), the nucleic acid probes may be positioned in the same region, or in different regions, on the surface. For instance, a first nucleic acid probe may be immobilized to a first region, a second nucleic acid probe may be immobilized to a second region, a third nucleic acid probe may be immobilized to a third region, a fourth nucleic acid probe may be immobilized to a fourth region, a fifth nucleic acid probe may be immobilized to a fifth region, etc.

Examples of surfaces include a wide variety of organic and inorganic polymers, as well as other materials, both natural and synthetic. Specific, non-limiting examples of solid surfaces include nitrocellulose, nylon, glass, fused silica, diazotized membranes (paper or nylon), silicones, cellulose, and cellulose acetate. In addition, plastics such as polyethylene, polypropylene, polystyrene, and the like can be used. Other materials which may be employed include paper, ceramics, metals, metalloids, semiconductive materials, cermets, or the like. In addition, substances that form gels can be used. Such materials include proteins (e.g., gelatins), lipopolysaccharides, silicates, agarose and polyacrylamides. Where the solid surface is porous, various pore sizes may be employed depending upon the nature of the system.

In one set of embodiments, the surface is the surface of an array, such as a microarray. Those of ordinary skill in the art will be familiar with the operation and use of arrays, i.e., a surface having a collection of elements or “spots,” which may be used to immobilize one or more compounds such as nucleic acid probes, as discussed in greater detail below. The elements on the substrate may be arranged in any suitable arrangement, for example, in a rectangular grid. The elements may be chosen to possess, or are chemically derivatized to possess, at least one reactive chemical group that can be used for further attachment chemistry, e.g., for attachment of a nucleic acid and/or a nucleic acid probe to the surface of the array. Such attachment may be covalent or non-covalent. There may also be optional molecular linkers interposed between the substrate and the reactive chemical groups used for molecular attachment.

Arrays on substrates with much lower fluorescence than membranes, such as glass, quartz, or small beads, can achieve much better sensitivity in certain embodiments. For example, elements of various sizes, ranging from the about 1 mm diameter down to about 1 micrometer can be used with these materials. Small array members containing small amounts of concentrated target DNA are conveniently used for high complexity comparative hybridizations since the total amount of probe available for binding to each element will be limited. Thus, it may be advantageous in certain embodiments to have small array members that contain a small amount of concentrated target DNA so that the signal that is obtained is highly localized and bright. Such small array members are typically used in arrays with densities greater than, e.g., about 10⁴/cm². Relatively simple approaches capable of quantitative fluorescent imaging of 1 cm² areas have been described that permit acquisition of data from a large number of members in a single image (see, e.g., Wittrup et al. Cytometry 16:206-213 (1994)).

The substrate of the array or other surface may be formed in essentially any shape. In one set of embodiments, the substrate has at least one surface which is substantially planar. However, in other embodiments, the substrate may also include indentations, protuberances, steps, ridges, terraces, or the like. The substrate may be formed from any suitable material, depending upon the application. For example, the substrate may be a silicon-based chip or a glass slide. Other suitable substrate materials for the arrays of the present invention include, but are not limited to, glasses, ceramics, plastics, metals, alloys, carbon, agarose, silica, quartz, cellulose, polyacrylamide, polyamide, polyimide, and gelatin, as well as other polymer supports or other solid-material supports. Polymers that may be used in the substrate include, but are not limited, to, polystyrene, poly(tetra)fluoroethylene (PTFE), polyvinylidenedifluoride, polycarbonate, polymethylmethacrylate, polyvinylethylene, polyethyleneimine, polyoxymethylene (POM), polyvinylphenol, polylactides, polymethacrylimide (PMI), polyalkenesulfone (PAS), polypropylene, polyethylene, polyhydroxyethylmethacrylate (HEMA), polydimethylsiloxane, polyacrylamide, polyimide, various block co-polymers, etc. Additional examples are discussed below.

The nucleic acids and/or the nucleic acid probes may be immobilized relative to a surface, e.g., the surface of an array, using any suitable technique known to those of ordinary skill in the art, for example, via chemical attachment (e.g., via covalent bonding), via one or more linkers bonded to the surface of the array (to which a nucleic acid or nucleic acid probe can bind), via non-covalent interactions, etc. In one set of embodiments, a linker may comprise one or more nucleic acids, and in some cases, at least a portion of the linker may comprise a hybridization region that is substantially complementary to a portion of a nucleic acid or a nucleic acid probe. For example, in one embodiment, the linker comprises a hybridization region that is substantially complementary to a tag sequence on a nucleic acid probe. If more than one nucleic acid probe is used, e.g., in an assay, the linkers may each comprise the same or different hybridization regions, for example, such that a first nucleic acid probe is able to bind a first linker (but not a second linker) and a second nucleic acid probe is able to bind the second linker (but not the first linker). Such discrimination may be achieved, for example, by using different tag sequences within the various nucleic acid probes, and such different tag sequences may be arbitrarily chosen in some instances. If an array is used, the linkers may be in the same or different elements or spots within the array.

The nucleic acids and/or the nucleic acid probes may be attached to surface before an assay is performed using the nucleic acids and/or nucleic acid probes, during, or afterwards. For example, in one embodiment, one or more nucleic acid probes may be immobilized relative to a surface, for instance, to one or more elements of an array, and subsequently exposed to one or more target nucleic acids to be probed. Hybridization of the nucleic acids and the nucleic acid probes may result in a number of nucleic acid-nucleic acid probe hybrids immobilized relative to the surface. The hybrids are then exposed to one or more restriction endonucleases, and the cleavage state of the hybrids can then be determined, e.g., whether the hybrids, or portions of the hybrids, remains immobilized relative to the surface.

Multiple assays may be performed using the same substrate, e.g., multiple assays for determining multiple allele types. For example, a surface may comprise a first CGH probe, a first set of allele nucleic acid probes, and a second set of allele nucleic acid probes, where each of the sets of allele nucleic acid probes may be used to determine a different allele of a target nucleic acid, for example, two different SNPs on the target nucleic acid. As another example, a surface may be used for assays in which more than one target nucleic acid is studied. For instance, the surface may comprise a first CGH probe and a first set of allele nucleic acid probes that a first target nucleic acid probe may associate with, and a second CGH probe and a second set of allele nucleic acid probes that a second target nucleic acid probe may associate with.

Another aspect of the invention is generally directed to a kit. A “kit,” as used herein, typically defines a package including one or more of the compositions of the invention, and/or other compositions associated with the invention, for example, one or more nucleic acid probes as previously described. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, modifying, assembling, storing, packaging, preparing, mixing, diluting, and/or preserving the compositions components for a particular use.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, assembly, storage, packaging, and/or preparation of the compositions and/or other compositions associated with the kit. In some cases, the instructions may also include instructions, for example, for a particular use. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

The kits may also comprise containers, each with one or more of the various reagents and/or compositions. The kits may also include a collection of immobilized oligonucleotide targets, e.g., one or more arrays of targets, and reagents employed in genomic template and/or labeled probe production, e.g., a highly processive polymerase, exonuclease resistant primers, random primers, buffers, the appropriate nucleotide triphosphates (e.g. dATP, dCTP, dGTP, dTTP), DNA polymerase, labeling reagents, e.g., labeled nucleotides, and the like. Where the kits are specifically designed for use in CGH applications, the kits may further include labeling reagents for making two or more collections of distinguishably labeled nucleic acids according to the subject methods, an array of target nucleic acids, hybridization solution, etc.

The following documents are incorporated herein by reference: U.S. patent application Ser. No. 10/448,298, filed May 28, 2003, entitled “Comparative Genomic Hybridization Assays using Immobilized Oligonucleotide Targets with Initially Small Sample Sizes and Compositions for Practicing the Same,” by M. T. Barrett, et al./, published as U.S. Patent Application Publication No. 2004/0241658 on Dec. 2, 2004; and International Patent Application No. PCT/US2003/041047, filed Dec. 22, 2003, entitled “Comparative Genomic Hybridization Assays using Immobilized Oligonucleotide Features and Compositions for Practicing the Same,” by L. K. Bruhn, et al., published as WO 2004/058945 A2 on Jul. 15, 2004.

Unless defined otherwise, all technical and scientific terms used herein have the same meaning as commonly understood by one of ordinary skill in the art to which this invention belongs. Still, certain terms are defined below for the sake of clarity and ease of reference.

The term “sample,” as used herein, relates to a material or mixture of materials, typically, although not necessarily, in fluid form, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

When two items are “associated” with one another, they are provided in such a way that it is apparent one is related to the other such as where one references the other. For example, an array identifier can be associated with an array by being on the array assembly (such as on the substrate or a housing) that carries the array or on or in a package or kit carrying the array assembly.

“Stably attached” or “stably associated with” means an item's position remains substantially constant.

“Contacting” means to bring or put together. As such, a first item is contacted with a second item when the two items are brought or put together, e.g., by touching them to each other.

“Depositing” means to position, place an item at a location, or otherwise cause an item to be so positioned or placed at a location. Depositing includes contacting one item with another. Depositing may be manual or automatic, e.g., “depositing” an item at a location may be accomplished by automated robotic devices.

The term “biomolecule” means any organic or biochemical molecule, group or species of interest that may be formed in an array on a substrate surface. Non-limiting examples of biomolecules include peptides, proteins, amino acids, and nucleic acids.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source. For example, a “biopolymer” may include DNA (including cDNA), RNA, oligonucleotides, and PNA and other polynucleotides as described in U.S. Pat. No. 5,948,902, incorporated herein by reference. A “biomonomer” refers to a single unit, which can be linked with the same or other biomonomers to form a biopolymer (e.g., a single amino acid or nucleotide with two linking groups, one or both of which may have removable protecting groups). A biomonomer fluid or biopolymer fluid references a liquid containing either a biomonomer or biopolymer, respectively (typically in solution).

The term “peptide,” as used herein, refers to any compound produced by amide formation between an alpha-carboxyl group of one amino acid and an alpha-amino group of another group. The term “oligopeptide,” as used herein, refers to peptides with fewer than about 10 to 20 residues, i.e., amino acid monomeric units. As used herein, the term “polypeptide” refers to peptides with more than 10 to 20 residues. The term “protein,” as used herein, refers to polypeptides of specific sequence of more than about 50 residues.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, epsilon-nicotinoyl-lysine, isonipecotic acid, tetrahydroisoquinoleic acid, alpha acid, sarcosine, citrulline, cysteic acid, t-butylglycine, t-butylalanine, phenylglycine, cyclohexylalanine, beta-alanine, 4-aminobutyric acid, and the like.

The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the substrate.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art, synthesis of nucleic acids of this type may utilize, in some cases, an initial substrate-bound monomer that is generally used as a building-block in a multi-step synthesis procedure to form a complete nucleic acid.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include, but are non limited to, deoxyribonucleotides (DNA), ribonucleotides (RNA), or other polynucleotides which are C-glycosides of a purine or pyrimidine base. The oligomer may be defined by, for example, about 2-500 monomers, about 10-500 monomers, or about 50-250 monomers.

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. The terms “ribonucleic acid” and “RNA,” as used herein, refer to a polymer comprising ribonucleotides. The terms “deoxyribonucleic acid” and “DNA,” as used herein, mean a polymer comprising deoxyribonucleotides. The term “oligonucleotide” as used herein denotes single stranded nucleotide multimers of from about 10 to 200 nucleotides and up to about 500 nucleotides in length. For instance, the oligonucleotide may be greater than about 60 nucleotides, greater than about 100 nucleotides or greater than about 150 nucleotides.

As used herein, a “target nucleic acid sample” or a “target nucleic acid” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or a “reference nucleic acid” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, alkylated riboses, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. Generally, as used herein, the terms “oligonucleotide” and “polynucleotide” are used interchangeably. Further, generally, the term “nucleic acid” or “nucleic acid molecule” also encompasses oligonucleotides and polynucleotides.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements, and amplification of any subchromosomal region or DNA sequence. In certain embodiments, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

The “genomic source” is the source of the initial nucleic acids from which the nucleic acid probes are produced, e.g., as a template in the labeled nucleic acid protocols described in greater detail herein. The genomic source may be prepared using any convenient protocol. In some embodiments, the genomic source is prepared by first obtaining a starting composition of genomic DNA, e.g., a nuclear fraction of a cell lysate, where any convenient means for obtaining such a fraction may be employed and numerous protocols for doing so are well known in the art. The genomic source is, in certain embodiments, genomic DNA representing the entire genome from a particular organism, tissue or cell type. A given initial genomic source may be prepared from a subject, for example a plant or an animal that is suspected of being homozygous or heterozygous for a deletion or amplification of a genomic region. In certain embodiments, the average size of the constituent molecules that make up the initial genomic source typically have an average size of at least about 1 Mb, where a representative range of sizes is from about 50 to about 250 Mb or more, while in other embodiments, the sizes may not exceed about 1 MB, such that the may be about 1 Mb or smaller, e.g., less than about 500 kb, etc.

If a surface-bound nucleic acid or probe “corresponds to” a chromosome, the polynucleotide usually contains a sequence of nucleic acids that is unique to that chromosome. Accordingly, a surface-bound polynucleotide that corresponds to a particular chromosome usually specifically hybridizes to a labeled nucleic acid made from that chromosome, relative to labeled nucleic acids made from other chromosomes. Array elements, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

The terms “hybridize” or “hybridization,” as is known to those of ordinary skill in the art, refer to the binding or duplexing of a nucleic acid molecule to a particular nucleotide sequence under suitable conditions, e.g., under stringent conditions. “Hybridizing” and “binding,” with respect to polynucleotides, are used interchangeably. The term “stringent conditions” (or “stringent hybridization conditions”) as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent conditions are the summation or combination (totality) of both hybridization and wash conditions.

Stringent conditions (e.g., as in array, Southern or Northern hybridizations) may be sequence dependent, and are often different under different experimental parameters. Stringent conditions that can be used to hybridize nucleic acids include, for instance, hybridization in a buffer comprising 50% formamide, 5×SSC (salt, sodium citrate), and 1% SDS at 42° C., or hybridization in a buffer comprising 5×SSC and 1% SDS at 65° C., both with a wash of 0.2×SSC and 0.1% SDS at 65° C. Other examples of stringent conditions include a hybridization in a buffer of 40% formamide, 1 M NaCl, and 1% SDS at 37° C., and a wash in 1×SSC at 45° C. In another example, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional examples of stringent conditions include hybridization at 60° C. or higher and 3×SSC (450 mM sodium chloride/45 mM sodium citrate) or incubation at 42° C. in a solution containing 30% formamide, 1 M NaCl, 0.5% sodium lauryl sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to another nucleic acid (for example, when a nucleic acid has hybridized to a nucleic acid probe). Wash conditions used to identify nucleic acids may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. The terms “high stringency conditions” or “highly stringent hybridization conditions,” as previously described, generally refers to conditions that are compatible to produce complexes between complementary binding members, i.e., between immobilized probes and complementary sample nucleic acids, but which does not result in any substantial complex formation between non-complementary nucleic acids (e.g., any complex formation which cannot be detected by normalizing against background signals to interfeature areas and/or control regions on the array).

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Additional hybridization methods are described in references describing CGH techniques (Kallioniemi et al., Science 1992; 258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, e.g., Gall et al. Meth. Enzymol. 1981; 21:470-480 and Angerer et al., In Genetic Engineering: Principles and Methods, Setlow and Hollaender, Eds. Vol 7, pgs 43-65 (Plenum Press, New York 1985). See also U.S. Pat. Nos. 6,335,167, 6,197,501, 5,830,645, and 5,665,549, the disclosures of which are herein incorporated by reference.

The phrases “nucleic acid molecule bound to a surface of a solid support,” “probe bound to a solid support,” “probe immobilized with respect to a surface,” “target bound to a solid support,” or “polynucleotide bound to a solid support” (and similar terms) generally refer to a nucleic acid molecule (e.g., an oligonucleotide or polynucleotide) or a mimetic thereof (e.g., comprising at least one PNA, UNA, and/or LNA monomer) that is immobilized on the surface of a solid substrate, where the substrate can have a variety of configurations, e.g., including, but not limited to, planar substrates, non-planar substrate, a sheet, bead, particle, slide, wafer, web, fiber, tube, capillary, microfluidic channel or reservoir, or other structure. The solid support may be porous or non-porous. In certain embodiments, collections of nucleic acid molecules are present on a surface of the same support, e.g., in the form of an array, which can include at least about two nucleic acid molecules. The two or more nucleic acid molecules may be identical or comprise a different nucleotide base composition.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. The term “feature” is used interchangeably herein, in this context, with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids.

In the broadest sense, the arrays of many embodiments are arrays of polymeric binding agents, where the polymeric binding agents may be any one or more of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5″ terminus). In some cases, the arrays are arrays of polypeptides, e.g., proteins or fragments thereof.

An “array” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain.

The term “substrate” as used herein refers to a surface upon which marker molecules or probes, e.g., an array, may be adhered. Glass slides are the most common substrate for biochips, although fused silica, silicon, plastic, and other materials are also suitable. The substrate may be formed in essentially any shape. In one set of embodiments, the substrate has at least one surface which is substantially planar. However, in other embodiments, the substrate may also include indentations, protuberances, steps, ridges, terraces, or the like. The substrate may be formed from any suitable material, depending upon the application. For example, the substrate may be a silicon-based chip or a glass slide. Other suitable substrate materials for the arrays of the present invention include, but are not limited to, glasses, ceramics, plastics, metals, alloys, carbon, agarose, silica, quartz, cellulose, polyacrylamide, polyamide, polyimide, and gelatin, as well as other polymer supports or other solid-material supports. Polymers that may be used in the substrate include, but are not limited to, polystyrene, poly(tetra)fluoroethylene (PTFE), polyvinylidenedifluoride, polycarbonate, polymethylmethacrylate, polyvinylethylene, polyethyleneimine, polyoxymethylene (POM), polyvinylphenol, polylactides, polymethacrylimide (PMI), polyalkenesulfone (PAS), polypropylene, polyethylene, polyhydroxyethylmethacrylate (HEMA), polydimethylsiloxane, polyacrylamide, polyimide, various block co-polymers, etc.

Any given substrate may carry any number of oligonucleotides on a surface thereof. In some cases, one, two, three, four, or more arrays may be disposed on a surface of the substrate. Depending upon the use, any or all of the arrays may be the same or different from one another and each may contain multiple spots, or elements or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from about 10 micrometers to 1.0 cm. In other embodiments each feature may have a width in the range of 1.0 micrometers to 1.0 mm, 5.0 micrometers to 500 micrometers, 10 micrometers to 200 micrometers, etc. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas may be present in some embodiments which do not carry any oligonucleotide (or other biopolymer or chemical moiety of a type of which the features are composed). Such interfeature areas may be present where the arrays are formed by processes involving drop deposition of reagents but may not be present when, for example, light directed synthesis fabrication processes are used. It will be appreciated though, that the interfeature areas, when present, could be of various sizes and configurations.

The substrate may have thereon a pattern of locations (or elements) (e.g., rows and columns) or may be unpatterned or comprise a random pattern. The elements may each independently be the same or different. For example, in certain cases, at least about 25% of the elements are substantially identical (e.g., comprise the same sequence composition and length). In certain other cases, at least 50% of the elements are substantially identical, or at least about 75% of the elements are substantially identical. In certain cases, some or all of the elements are completely or at least substantially identical. For instance, if nucleic acids are immobilized on the surface of a solid substrate, at least about 25%, at least about 50%, or at least about 75% of the oligonucleotides may have the same length, and in some cases, may be substantially identical.

An “array layout” or “array characteristics” refers to one or more physical, chemical or biological characteristics of the array, such as positioning of some or all the features within the array and on a substrate, one or more dimensions of the spots or elements, or some indication of an identity or function (for example, chemical or biological) of a moiety at a given location, or how the array should be handled (for example, conditions under which the array is exposed to a sample, or array reading specifications or controls following sample exposure).

Each array may cover an area of less than 100 cm², or even less than 50 cm², 10 cm², 1 cm², 0.5 cm², or 0.1 cm² In certain embodiments, the substrate carrying the one or more arrays will be shaped as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. In some cases, the array will have a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. In some instances, with arrays that are read by detecting fluorescence, the substrate may be of a material that emits low fluorescence upon illumination with the excitation light. Additionally, in some cases the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident thereon, as may be measured across the entire integrated spectrum of such illuminating light or alternatively at 532 nm or 633 nm.

In certain embodiments of particular interest, in situ prepared arrays are employed. In situ prepared oligonucleotide arrays, e.g., nucleic acid arrays, may be characterized by having surface properties of the substrate that differ significantly between the feature and interfeature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water, as known in the art and further described in copending application Ser. No. 10/449,838, the disclosure of which is herein incorporated by reference. Other features of in situ prepared arrays that make such array formats of particular interest in certain embodiments of the present invention include, but are not limited to: feature density, oligonucleotide density within each feature, feature uniformity, low intra-feature background, low interfeature background, e.g., due to hydrophobic interfeature regions, fidelity of oligonucleotide elements making up the individual features, array/feature reproducibility, and the like. The above benefits of in situ produced arrays assist in maintaining adequate sensitivity while operating under stringency conditions required to accommodate highly complex samples.

In certain embodiments, a nucleic acid sequence may be present as a composition of multiple copies of the nucleic acid molecule on the surface of the array, e.g., as a spot or element on the surface of the substrate. The spots may be present as a pattern, where the pattern may be in the form of organized rows and columns of spots, e.g., a grid of spots, across the substrate surface, a series of curvilinear rows across the substrate surface, e.g., a series of concentric circles or semi-circles of spots, or the like. The density of spots present on the array surface may vary, for example, at least about 10, at least about 100 spots/cm², at least about 1,000 spots/cm², or at least about 10,000 spots/cm². In other embodiments, however, the elements are not arranged in the form of distinct spots, but may be positioned on the surface such that there is substantially no space separating one element from another.

In certain aspects, in constructing arrays, both coding and non-coding genomic regions are included as probes, whereby “coding region” refers to a region comprising one or more exons that is transcribed into an mRNA product and from there translated into a protein product, while by non-coding region is meant any sequences outside of the exon regions, where such regions may include regulatory sequences, e.g., promoters, enhancers, untranslated but transcribed regions, introns, origins of replication, telomeres, etc. In certain embodiments, one can have at least some of the probes directed to non-coding regions and others directed to coding regions. In certain embodiments, one can have all of the probes directed to non-coding sequences and such sequences can, optionally, be all non-transcribed sequences (e.g., intergenic regions including regulatory sequences such as promoters and/or enhancers lying outside of transcribed regions).

In certain aspects, an array may be optimized for one type of genome scanning application compared to another, for example, the array can be enriched for intergenic regions compared to coding regions for a location analysis application. In some embodiments, at least 5% of the polynucleotide probes on the solid support hybridize to regulatory regions of a nucleotide sample of interest while other embodiments may have at least 30% of the polynucleotide probes on the solid support hybridize to exonic regions of a nucleotide sample of interest. In yet other embodiments, at least 50% of the polynucleotide probes on the solid support hybridize to intergenic regions (e.g., non-coding regions which exclude introns and untranslated regions, i.e, comprise non-transcribed sequences) of a nucleotide sample of interest.

In certain aspects, probes on the array represent random selection of genomic sequences (e.g., both coding and noncoding). However, in other aspects, particular regions of the genome are selected for representation on the array, e.g., such as CpG islands, genes belonging to particular pathways of interest or whose expression and/or copy number are associated with particular physiological responses of interest (e.g., disease, such a cancer, drug resistance, toxological responses and the like). In certain aspects, where particular genes are identified as being of interest, intergenic regions proximal to those genes are included on the array along with, optionally, all or portions of the coding sequence corresponding to the genes. In one aspect, at least about 100 bp, 500 bp, 1,000 bp, 5,000 bp, 10,000 kb or even 100,000 kb of genomic DNA upstream of a transcriptional start site is represented on the array in discrete or overlapping sequence probes. In certain aspects, at least one probe sequence comprises a motif sequence to which a protein of interest (e.g., such as a transcription factor) is known or suspected to bind.

In certain aspects, repetitive sequences are excluded as probes on the arrays. However, in another aspect, repetitive sequences are included.

The choice of nucleic acids to use as probes may be influenced by prior knowledge of the association of a particular chromosome or chromosomal region with certain disease conditions. Int. Pat. Apl. WO 93/18186 provides a list of exemplary chromosomal abnormalities and associated diseases, which are described in the scientific literature. Alternatively, whole genome screening to identify new regions subject to frequent changes in copy number can be performed using the methods of the present invention discussed further below.

In some embodiments, previously identified regions from a particular chromosomal region of interest are used as probes. In certain embodiments, the array can include probes which “tile” a particular region (e.g., which have been identified in a previous assay or from a genetic analysis of linkage), by which is meant that the probes correspond to a region of interest as well as genomic sequences found at defined intervals on either side, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled array tailored to the initially identified region to further assay the region at a higher resolution, e.g., in an iterative protocol.

In certain aspects, the array includes probes to sequences associated with diseases associated with chromosomal imbalances for prenatal testing. For example, in one aspect, the array comprises probes complementary to all or a portion of chromosome 21 (e.g., Down's syndrome), all or a portion of the X chromosome (e.g., to detect an X chromosome deficiency as in Turner's Syndrome) and/or all or a portion of the Y chromosome Klinefelter Syndrome (to detect duplication of an X chromosome and the presence of a Y chromosome), all or a portion of chromosome 7 (e.g., to detect William's Syndrome), all or a portion of chromosome 8 (e.g., to detect Langer-Giedon Syndrome), all or a portion of chromosome 15 (e.g., to detect Prader-Willi or Angelman's Syndrome, all or a portion of chromosome 22 (e.g., to detect Di George's syndrome).

Other “themed” arrays may be fabricated, for example, arrays including whose duplications or deletions are associated with specific types of cancer (e.g., breast cancer, prostate cancer and the like). The selection of such arrays may be based on patient information such as familial inheritance of particular genetic abnormalities. In certain aspects, an array for scanning an entire genome is first contacted with a sample and then a higher-resolution array is selected based on the results of such scanning. Themed arrays also can be fabricated for use in gene expression assays, for example, to detect expression of genes involved in selected pathways of interest, or genes associated with particular diseases of interest.

In one embodiment, a plurality of probes on the array is selected to have a duplex Tₘ within a predetermined range. For example, in one aspect, at least about 50% of the probes have a duplex Tₘ within a temperature range of about 75° C. to about 85° C. In one embodiment, at least 80% of said polynucleotide probes have a duplex Tₘ within a temperature range of about 75° C. to about 85° C., within a range of about 77° C. to about 83° C., within a range of from about 78° C. to about 82° C. or within a range from about 79° C. to about 82° C. In one aspect, at least about 50% of probes on an array have range of Tₘ's of less than about 4° C., less then about 3° C., or even less than about 2° C., e.g., less than about 1.5° C., less than about 1.0° C. or about 0.5° C.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In still other aspects, probes on the array comprise at least coding sequences. In one aspect, probes represent sequences from an organism such as Drosophila melanogaster, Caenorhabditis elegans, yeast, zebrafish, a mouse, a rat, a domestic animal, a companion animal, a primate, a human, etc. In certain aspects, probes representing sequences from different organisms are provided on a single substrate, e.g., on a plurality of different arrays.

In some embodiments, the array may be referred to as addressable. An array is “addressable” when it has multiple regions of different moieties (e.g., different nucleic acids) such that a region (i.e., an element or “spot” of the array) at a particular predetermined location (i.e., an “address”) on the array may be used to detect a particular target or class of targets (although an element may incidentally detect non-targets of that element). In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., nucleic acid molecules, to be evaluated by binding with the other).

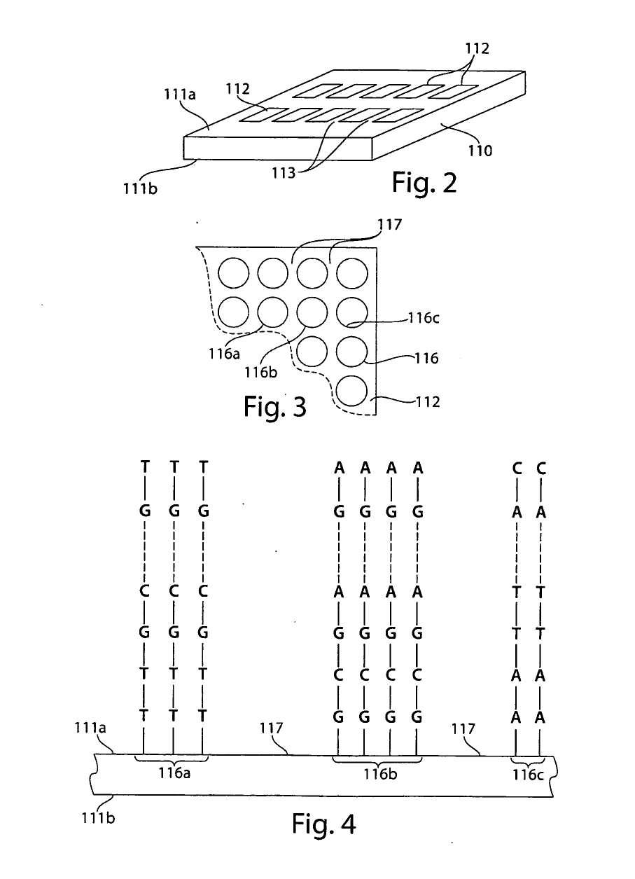

An example of an array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of oligomers, e.g., in the form of polynucleotides, and specifically oligonucleotides. As mentioned above, all of the features 116 may be different, or some or all could be the same. The interfeature areas 117 could be of various sizes and configurations. Each feature carries a predetermined oligomer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In the case of an array in the context of the present application, the “target” may be referenced as a moiety in a mobile phase (typically fluid), to be detected by “probes” which are bound to the substrate at the various regions.

A “scan region” refers to a contiguous (preferably, rectangular) area in which the array spots or elements of interest, as discussed above, are found. For example, the scan region may be that portion of the total area illuminated from which resulting fluorescence is detected and recorded. For the purposes of this invention, the scan region includes the entire area of the slide scanned in each pass of the lens, between the first element of interest, and the last element of interest, even if there are intervening areas which lack elements of interest. An “array layout” refers to one or more characteristics of the features, such as element positioning on the substrate, one or more feature dimensions, and an indication of a moiety at a given location.

In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

The arrays can be fabricated using drop deposition from pulsejets of either oligonucleotide precursor units (such as monomers) in the case of in situ fabrication, or the previously obtained oligonucleotide. Such methods are described in detail in, for example, in U.S. Pat. Nos. 6,242,266, 6,232,072, 6,180,351, 6,171,797, or 6,323,043, or in U.S. patent application Ser. No. 09/302,898, filed Apr. 30, 1999, and the references cited therein. These references are each incorporated herein by reference. Other drop deposition methods can be used for fabrication, as previously described herein.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein and thus can also be referred to as a “location analysis array” or an “array for ChIP-chip analysis.” In certain aspects, a CGH array provides probes for screening or scanning a genome of an organism and comprises probes from a plurality of regions of the genome.

In using an array made by the method of the present invention, the array will be exposed in certain embodiments to a sample (for example, a fluorescently labeled target nucleic acid molecule) and the array then read. Reading of the array may be accomplished, for instance, by illuminating the array and reading the location and intensity of resulting fluorescence at various locations of the array (e.g., at each spot or element) to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. Pat. Nos. 6,756,202 or 6,406,849, each incorporated herein by reference.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one set of embodiments, the control target molecules in a population are present at a level comparable to a haploid amount of a gene represented in the target population. In other embodiments, the control target molecules are present at a level comparable to a diploid amount of a gene. In still other embodiments, the control target molecules are present at a level that is different from a haploid or diploid amount of a gene represented in the target population. The relative proportions of complexes formed labeled with the first label vs. the second label can be used to evaluate relative copy numbers of targets found in the two samples.

In certain embodiments, test and reference populations of nucleic acids may be applied separately to separate but identical arrays (e.g., having identical probe molecules) and the signals from each array can be compared to determine relative copy numbers of the nucleic acids in the test and reference populations.

Arrays may also be read by any other method or apparatus than the foregoing, with other reading methods, including other optical techniques (for example, detecting chemiluminescent or electroluminescent labels) or electrical techniques (where each feature is provided with an electrode to detect hybridization at that feature in a manner disclosed in, e.g., U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition).

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

The following examples are intended to illustrate certain embodiments of the present invention, but do not exemplify the full scope of the invention.

### EXAMPLE 1

This example illustrates combined CGH and allele detection probe sets of certain embodiments of the invention. Table I illustrates two sets of longer (60-mer) CGH probes and shorter allele detection probes corresponding to each of the 4 possible allelic variants for two polymorphic loci in the human genome (with the variant nucleotide indicated by underlining). Each CGH probe is bounded by a restriction enzyme recognition site at the 5′ end which terminates the allele-specific extension products.

TABLE 1
Set #1

| | | |
|---|---|---|
| SNP ID: rs11785668 | | |
| CGH probe | CTGTAAGATAATGTTGCTTTCTTATCCCAGTGATCACCTGCCAAATGAATAAGACAACAA | (SEQ ID NO:1) |
| Allelic probe A | AGTGATCACCTGCCAAATGAATAAGACAACAA | (SEQ ID NO:2) |
| Allelic probe G | GGTGATCACCTGCCAAATGAATAAGACAACAA | (SEQ ID NO:3) |
| Allelic probe T | TGTGATCACCTGCCAAATGAATAAGACAACAA | (SEQ ID NO:4) |
| Allelic probe C | CGTGATCACCTGCCAAATGAATAAGACAACAA | (SEQ ID NO:5) |
| Set #2 | | |
| SNP ID: rs4606794 | | |
| CGH probe | ACGTAGGAAAATGTGAAATGTTCCTGTTCTTACATAAAAGAACTCTCAGAAAATACCCGT | (SEQ ID NO:6) |
| Allelic probe A | ATACATAAAAGAACTCTCAGAAAATACCCGT | (SEQ ID NO:7) |
| Allelic probe G | GTACATAAAAGAACTCTCAGAAAATACCCGT | (SEQ ID NO:8) |
| Allelic probe T | TTACATAAAAGAACTCTCAGAAAATACCCGT | (SEQ ID NO:9) |
| Allelic probe C | CTACATAAAAGAACTCTCAGAAAATACCCGT | (SEQ ID NO:10) |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. All definitions, as defined and used herein, should be understood to control over dictionary definitions, definitions in documents incorporated by reference, and/or ordinary meanings of the defined terms.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. In this specification and the appended claims, the singular forms “a,” “an” and “the” include plural reference unless the context clearly dictates otherwise.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

“Optional” or “optionally,” as used herein, means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

It should also be understood that, unless clearly indicated to the contrary, in any methods claimed herein that include more than one step or act, the order of the steps or acts of the method is not necessarily limited to the order in which the steps or acts of the method are recited.

All publications mentioned herein are incorporated herein by reference for the purpose of describing and disclosing the invention components that are described in the publications that might be used in connection with the presently described invention.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A solid support, comprising:
in a first region, a first nucleic acid; and
in second, third, fourth, and fifth regions, respectively, second, third, fourth, and fifth nucleic acids, each having a length that is shorter than the length of the first nucleic acid, each being immobilized relative to the solid support at a first end of each respective nucleic acid, each of the second, third, fourth, and fifth nucleic acids being substantially identical, wherein no two of the second, third, fourth, and fifth nucleic acids ends with identical terminal nucleotides.

2. The solid support of claim 1, wherein each of the second, third, fourth, and fifth nucleic acids is less than about two-thirds of the length of the first nucleic acid.

3. The solid support of claim 1, wherein each of the second, third, fourth and fifth nucleic acids are less than about one-half of the length of the first nucleic acid.

4. The solid support of claim 1, wherein the first nucleic acid has a length of between 40 nucleotides and 200 nucleotides, inclusive.

5. The solid support of claim 4, wherein the first nucleic acid has between 45 nucleotides and 60 nucleotides, inclusive.

6. The solid support of claim 1, wherein each of the second, third, fourth and fifth nucleic acids has between 20 nucleotides and 50 nucleotides, inclusive.

7. The solid support of claim 6, wherein each of the second, third, fourth, and fifth nucleic acids has between 20 nucleotides and 35 nucleotides, inclusive.

8. The solid support of claim 1, wherein each of the second, third, fourth, and fifth nucleic acids are identical except for the terminal nucleotides.

9. The solid support of claim 1, wherein the solid support is a microarray.

10. The solid support of claim 1, further comprising a first detection entity immobilized relative to the first nucleic acid.

11. The solid support of claim 1, wherein each of the second, third, fourth, and fifth nucleic acids has a second detection entity immobilized relative thereto.

12. A method, comprising acts of:
providing a first nucleic acid;
providing at least three types of substantially identical nucleic acid probes, each having a length that is shorter than the length of the first nucleic acid no two of the at least three types of substantially identical nucleic acid probes ending with identical terminal nucleotides;
hybridizing a target nucleic acid to at least some of the nucleic acid probes, the target nucleic acid having a length greater than the length of the nucleic acid probes;
substantially complementarily extending one of the probe nucleic acids along the target nucleic acid, without substantially extending the length of the other probe nucleic acids by subjecting the probe nucleic acids to primer extension reaction conditions;
evaluating binding of the target nucleic acid and the first nucleic acid; and
evaluating binding of the target acid with the nucleic acid probes.

13. The method of claim 12, wherein the first nucleic acid has a length of between 40 nucleotides and 200 nucleotides, inclusive.

14. The method of claim 12, comprising providing at least four types of substantially identical nucleic acid probes, each having a length of between 20 nucleotides and 50 nucleotides, inclusive, no two of the at least four types of substantially identical nucleic acid probes ending with identical terminal nucleotides.

15. The method of claim 12, wherein each of the at least three types of nucleic acid probes are identical except for the terminal nucleotides.

16. The method of claim 12, comprising hybridizing the target nucleic acid to the first nucleic acid.

17. The method of claim 12, further comprising immobilizing a first detection entity with respect to the first nucleic acid.

18. The method of claim 12, comprising identifying a SNP of the target nucleic acid by determining association of the target acid with one of the at least three types of substantially identical nucleic acid probes.

19. A solid support, comprising:
in first, second, third, fourth, and fifth regions, respectively, first, second, third, fourth, and fifth nucleic acids, each of the second, third, fourth, and fifth nucleic acids being substantially identical and having between 20 nucleotides and 50 nucleotides, inclusive, wherein the first nucleic acid comprises a portion substantially identical to the second, third, fourth, and fifth nucleic acids.

20. A kit, comprising:
a first nucleic acid; and
second, third, fourth, and fifth nucleic acids, each having a length that is shorter than the length of the first nucleic acid, each of the second, third, fourth, and fifth nucleic acid being substantially identical, wherein no two of the second, third, fourth, and fifth nucleic acids ends with identical terminal nucleotides.
